(19) United States
(12) Patent Application Publication
(10) Pub. No.: US 2025/0110137 A1
(43) Pub. Date: Apr. 3, 2025

(54) Composition for Detecting Occult Blood in Excreta, and Preparation Method Therefor and Use Thereof

(71) Applicant: Shanghai Peige Hospital Management Co., Ltd., Shanghai (CN)

(72) Inventors: Xi Wang, Shanghai (CN); Weixue Guo, Shanghai (CN); Pingjun Ren, Shanghai (CN); Fei Xiao; Sai Wu, Shanghai (CN); Min Li, Shanghai (CN)

(73) Assignee: Shanghai Peige Hospital Management Co., Ltd., Shanghai (CN)

(21) Appl. No.: 18/727,859

(22) PCT Filed: Apr. 25, 2023
(86) PCT No.: PCT/CN2023/090486

§ 371 Date: Jul. 10, 2024

(30) Foreign Application Priority Data
Nov. 22, 2022 (CN) 202211466586.1

(51) Int. Cl. G01N 33/72; G01N 21/78

(52) U.S. Cl. CPC G01N 33/725; G01N 21/78; G01N 2333/805

(57) ABSTRACT

Provided in the present application are a composition for detecting occult blood in excreta, and a preparation method therefor and the use thereof. The composition comprises, in parts by weight, 20-80 parts of organic solvent, 0.01-50 parts of thickener, 0.01-2 parts of benzidine, 0.01-5 parts of peroxide, 0.001-0.5 parts of EDTA-2Na and 10-80 parts of water. The composition provided in the present application can quickly and effectively detect the occult blood condition of excreta, has fast color change, long color retention time and high sensitivity.

### TECHNICAL FIELD

The present application belongs to the field of disease detection, specifically relates to a composition for detecting occult blood in excretion, a preparation method therefor, and an application thereof, and in particular to a composition for detecting occult blood in excretion with high sensitivity, a preparation method and an application thereof.

### BACKGROUND

Having blood in excretion is a common pathological symptom. The diseases that may cause blood in feces include, but are not limited to: hemorrhoids, ulcerative colitis, upper gastrointestinal bleeding, lower gastrointestinal bleeding, blood system disease, anal fissure, gastrointestinal tumor, etc.; The diseases that may cause blood in urine include, but are not limited to: urinary system mucosal injury, urinary system stone, nephritis, urinary system tumor, etc. Digestive system bleeding and urinary system bleeding caused by the above diseases have two conditions: visible blood and occult blood. The blood visible to the naked eye in excretion is easily noticed, but the initial symptoms of most diseases are occult blood in excretion. When the excretion contains visible blood, the disease may already be quite severe, so the regular occult blood test can provide early screening for urinary and digestive diseases.

CN114354948A discloses a semi-quantitative method for detecting fecal occult blood, steps of the semi-quantitative method for detecting fecal occult blood are: a diluted sample is added dropwise to a sample well on a colloidal gold test card through a sample device, and after the preliminary result qualitatively indicating negative or positive, the qualitative result on the display is photographed and read; after the qualitative result is read, a certain amount of 3,3,5,5-tetramethylbenzidine indicator solution is added dropwise to the same sampling well, and then a certain amount of hydrogen peroxide solution is added dropwise; after a reaction period of the 3,3,5,5-tetramethylbenzidine indicator solution and the hydrogen peroxide solution dropwise added, the colloidal gold test card shows a semi-quantitative result, and the semi-quantitative result is photographed and read again. In this invention, the fecal occult blood test provides the semi-quantitative result additionally, the manual operation is reduced, and the labor workload is reduced, thereby overcoming the defects of the fecal occult blood test in clinical examination, greatly reducing the work of manual rechecking, and ensuring the accuracy of test results.

CN103175965A discloses a fecal occult blood test kit which comprises a fecal occult blood test strip or test card, a sample processing tube or a feces-sampling mixer, and a sampling stick; the test strip or test card comprises a detection area (T) which is coated with an anti-Hb1 monoclonal antibody, and a quality control area (C) which is coated with a sheep anti-mouse polyclonal antibody, and colloidal gold which is coated with an anti-Hb2 monoclonal antibody. The preparation method of the above kit comprises preparation of a treatment solution, preparation of a kit, preparation of a colloidal gold complex, gold-spraying, cutting, assembling, cutting, and packaging, etc. The fecal occult blood test kit has good specificity, high sensitivity, rapid and simple detection, and is not restricted by dietary or drugs.

Because the occult blood test can play an early screening effect on urinary and digestive diseases, how to provide an occult blood test method or reagent with easy detection and high sensitivity has become an urgent problem to be solved.

### SUMMARY

In view of the shortcomings of the related art, the present application provides a composition for detecting occult blood in excretion, a preparation method therefor, and an application thereof, in particular to a composition for detecting occult blood in excretion with high sensitivity, a preparation method and an application thereof. The composition provided in the present application can quickly and effectively detect occult blood in excretion, which has a fast color change speed, long color retention duration, and high sensitivity.

The present application adopts the following technical solutions.

In a first aspect, an embodiment of the present application provides a composition for detecting occult blood in excretion, and the composition comprises 20-80 parts by weight of an organic solvent, 0.01-50 parts by weight of a thickener, 0.01-2 parts by weight of benzidine, 0.01-5 parts by weight of peroxide, 0.001-0.5 parts by weight of EDTA-2Na, and 10-80 parts by weight of water.

The organic solvent can be 20 parts, 30 parts, 40 parts, 50 parts, 60 parts, 70 parts, or 80 parts, etc.; the thickener can be 0.01 parts, 0.1 parts, 0.5 parts, 1 part, 5 parts, 10 parts, 15 parts, 20 parts, 25 parts, 30 parts, 35 parts, 40 parts, 45 parts, or 50 parts, etc.; the benzidine can be 0.01 parts, 0.1 parts, 0.5 parts, 1 part, 1.5 parts, or 2 parts, etc.; the peroxide can be 0.01 parts, 0.1 parts, 0.5 parts, 1 parts, 1.5 parts, 2 parts, 2.5 parts, 3 parts, 3.5 parts, 4 parts, 4.5 parts, or 5 parts, etc.; the EDTA-2Na can be 0.001 parts, 0.01 parts, 0.1 parts, 0.2 parts, 0.3 parts, 0.4 parts, or 0.5 parts, etc., and the water can be 10 parts, 20 parts, 30 parts, 40 parts, 50 parts, 60 parts, 70 parts, or 80 parts, etc.; however, the part is not limited to the listed values, and other unlisted values within the numerical range are also applicable.

The composition can effectively detect occult blood in excretion due to the particularly selected components, which has a fast color change speed, long color retention duration, and high sensitivity.

In one embodiment, the composition comprises 40-60 parts by weight of the organic solvent, 10-40 parts by weight of the thickener, 0.5-1.5 parts by weight of the benzidine, 1-4 parts by weight of the peroxide, 0.1-0.3 parts by weight of the EDTA-2Na, and 30-60 parts by weight of the water.

In one embodiment, the organic solvent comprises any one or a combination of at least two of ethanol, benzene, toluene, pentane, hexane, methanol, ethyl ether, ethyl acetate, acetone, or carbon tetrachloride, such as a combination of ethanol and acetone, a combination of ethanol and toluene, or a combination of methanol and ethanol; however, the combination is not limited to the listed combinations, and other unlisted combinations within the combination range are also applicable.

In one embodiment, the organic solvent comprises a combination of ethanol and acetone.

In one embodiment, the thickener comprises any one or a combination of at least two of xanthan gum, guar gum, pectin, sodium polyacrylate, hydroxypropyl methyl cellulose, hydroxymethyl cellulose sodium, or hydroxymethyl cellulose, such as a combination of xanthan gum and guar gum, a combination of guar gum and pectin, or a combination of pectin and sodium polyacrylate; however, the combination is not limited to the listed combinations, and other unlisted combinations within the combination range are also applicable.

In one embodiment, the benzidine comprises any one or a combination of at least two of 4,4′-diaminobiphenyl, tetramethyl benzidine, or 3,3′-dihydroxybenzidine, such as a combination of 4,4′-diaminobiphenyl and tetramethyl benzidine, a combination of tetramethyl benzidine and 3,3′-dihydroxybenzidine, or a combination of 4,4′-diaminobiphenyl and 3,3′-dihydroxybenzidine; however, the combination is not limited to the listed combinations, and other unlisted combinations within the combination range are also applicable.

In one embodiment, the benzidine comprises a combination of 3,3′-dihydroxybenzidine and tetramethyl benzidine.

The combination of the above specific benzidine can further improve the technical effect of the composition, accelerate the color change speed, prolong the color retention duration, and further improve the detection sensitivity.

In one embodiment, the peroxide comprises inorganic peroxide and/or organic peroxide.

In one embodiment, the peroxide comprises a combination of inorganic peroxide and organic peroxide.

In one embodiment, the inorganic peroxide comprises any one or a combination of at least two of hydrogen peroxide, sodium percarbonate, potassium peroxide, calcium peroxide, or barium peroxide, such as a combination of hydrogen peroxide and sodium percarbonate, a combination of sodium percarbonate and potassium peroxide, or a combination of potassium peroxide and calcium peroxide; however, the combination is not limited to the listed combinations, and other unlisted combinations within the combination range are also applicable.

In one embodiment, the inorganic peroxide comprises hydrogen peroxide.

In one embodiment, the organic peroxide comprises any one or a combination of at least two of cumene hydroperoxide, diisopropylbenzene hydroperoxide, tert-amyl peroxy-2-ethylhexanoate, or tert-butyl peroxy-2-ethylhexanoate, such as a combination of cumene hydroperoxide and diisopropylbenzene hydroperoxide, a combination of diisopropylbenzene hydroperoxide and tert-amyl peroxy-2-ethylhexanoate, or a combination of tert-amyl peroxy-2-ethylhexanoate and tert-butyl peroxy-2-ethylhexanoate; however, the combination is not limited to the listed combinations, and other unlisted combinations within the combination range are also applicable.

In one embodiment, the organic peroxide comprises cumene hydroperoxide.

The above specific peroxide and combinations thereof can effectively improve the technical effect of the composition, accelerate the color change speed, prolong the color retention duration, and further improve the detection sensitivity.

In one embodiment, the composition further comprises 0.01-5 parts of a pH-adjusting agent by weight, such as 0.01 parts, 0.1 parts, 0.5 parts, 1 part, 1.5 parts, 2 parts, 2.5 parts, 3 parts, 3.5 parts, 4 parts, 4.5 parts, or 5 parts; however, the part is not limited to the listed values, and other unlisted values within the numerical range are also applicable.

In one embodiment, the pH-adjusting agent comprises any one of citric acid, acetic acid, sodium hydroxide, or hydrochloric acid.

In one embodiment, the composition further comprises 1-80 parts of starch by weight, such as 1 part, 10 parts, 20 parts, 30 parts, 40 parts, 50 parts, 60 parts, 70 parts, or 80 parts; however, the part is not limited to the listed values, and other unlisted values within the numerical range are also applicable.

In one embodiment, the starch comprises any one or a combination of at least two of corn starch, pea starch, mung bean starch, gelatinized starch, hydroxymethyl starch, crosslinked starch, maltodextrin, oxidized starch, esterified starch, or etherified starch, such as a combination of corn starch and pea starch, a combination of pea starch and mung bean starch, or a combination of mung bean starch and gelatinized starch; however, the combination is not limited to the listed combinations, and other unlisted combinations within the combination range are also applicable.

The above starch can be added according to the requirements of users to make the composition from liquid to solid, which is applicable to different scenarios.

In a second aspect, an embodiment of the present application provides a preparation method for the composition for detecting occult blood in excretion as described above, and the preparation method comprises the following steps:
(1) mixing benzidine, peroxide, and an organic solvent to obtain an organic phase;
(2) mixing a thickener, EDTA-2Na, and water to obtain an aqueous phase; and
(3) mixing the aqueous phase with the organic phase to obtain the composition for detecting occult blood in excretion;
steps (1) and (2) are in no particular order.

In one embodiment, in step (2), mixing the thickener, EDTA-2Na, and water to obtain the aqueous phase further comprises mixing with a pH-adjusting agent.

In one embodiment, in step (3), mixing the aqueous phase with the organic phase further comprises mixing with starch.

In a third aspect, an embodiment of the present application also provides an application of the composition for detecting occult blood in excretion as described above in the preparation of an occult blood detection device.

In one embodiment, the occult blood detection device comprises any one of a test paper, a test plate, a reagent kit, or a test tube.

Compared with the related art, the present application has the following beneficial effects.

The present application provides a composition for detecting occult blood in excretion, which can effectively detect occult blood in excretion by selecting a specific benzidine and peroxide, and has a fast color change speed, long color retention duration, and high sensitivity.

### DETAILED DESCRIPTION

In order to further illustrate the technical solutions adopted in the present application and their effects, the technical solutions of the present application are further described below in terms of optional examples of the present application, but the present application is not limited to the scope of the examples.

### EXAMPLE 1

This example provides a composition for detecting occult blood in excretion, and the preparing raw materials were as follows:

30 parts by weight of ethanol, 20 parts by weight of acetone, 25 parts by weight of xanthan gum, 0.5 parts by weight of 3,3′-dihydroxybenzidine, 0.5 parts by weight of tetramethylbenzidine, 1 part by weight of hydrogen peroxide, 1 part by weight of cumene hydroperoxide, 0.25 parts by weight of EDTA-2Na, 50 parts by weight of water, and 2.5 parts by weight of citric acid.

The preparation method is as follows:
(1) ethanol, 3,3′-dihydroxybenzidine, tetramethylbenzidine, hydrogen peroxide, and cumene hydroperoxide were mixed and stirred to obtain an organic phase;
(2) water, xanthan gum, EDTA-2Na, and citric acid were mixed to obtain an aqueous phase; and
(3) the aqueous phase and the organic phase were mixed and stirred to obtain the composition for detecting occult blood in excretion.

### EXAMPLE 2

This example provides a composition for detecting occult blood in excretion, and the preparing raw materials were as follows:

40 parts by weight of ethanol, 10 parts by weight of pectin, 0.5 parts by weight of tetramethylbenzidine, 1 part by weight of cumene hydroperoxide, 0.1 parts by weight of EDTA-2Na, 30 parts by weight of water, and 0.1 parts by weight of citric acid.

The preparation method referred to Example 1.

### EXAMPLE 3

This example provides a composition for detecting occult blood in excretion, and the preparing raw materials were as follows:

60 parts by weight of ethanol, 40 parts by weight of guar gum, 1.5 parts by weight of 4,4′-diaminobiphenyl, 4 parts by weight of hydrogen peroxide, 0.3 parts by weight of EDTA-2Na, 60 parts by weight of water, and 5 parts by weight of citric acid.

The preparation method referred to Example 1.

### EXAMPLE 4

This example provides a composition for detecting occult blood in excretion, and the preparing raw materials were as follows:

20 parts by weight of ethanol, 0.01 parts by weight of pectin, 0.01 parts by weight of tetramethylbenzidine, 0.01 parts by weight of cumene hydroperoxide, 0.001 parts by weight of EDTA-2Na, 10 parts by weight of water, and 0.1 parts by weight of citric acid.

The preparation method referred to Example 1.

### EXAMPLE 5

This example provides a composition for detecting occult blood in excretion, and the preparing raw materials were as follows:

80 parts by weight of ethanol, 50 parts by weight of guar gum, 2 parts by weight of 4,4′-diaminobiphenyl, parts by weight of hydrogen peroxide, 0.5 parts by weight of EDTA-2Na, 80 parts by weight of water, and 5 parts by weight of citric acid.

The preparation method referred to Example 1.

### EXAMPLE 6

This example provides a composition for detecting occult blood in excretion, wherein the preparing raw materials were the same as in Example 1 except that 3,3′-dihydroxybenzidine was not added and the resulting absence was made up by tetramethylbenzidine.

The preparation method referred to Example 1.

### EXAMPLE 7

This example provides a composition for detecting occult blood in excretion, wherein the preparing raw materials were the same as in Example 1 except that tetramethylbenzidine was not added and the resulting absence was made up by 3,3′-dihydroxybenzidine.

The preparation method referred to Example 1.

### EXAMPLE 8

This example provides a composition for detecting occult blood in excretion, wherein the preparing raw materials were the same as in Example 1 except that 3,3′-dihydroxybenzidine was replaced with an equivalent amount of 4,4′-diaminobiphenyl.

The preparation method referred to Example 1.

### EXAMPLE 9

This example provides a composition for detecting occult blood in excretion, wherein the preparing raw materials were the same as in Example 1 except that hydrogen peroxide was not added and the resulting absence was made up by cumene hydroperoxide.

The preparation method referred to Example 1.

### EXAMPLE 10

This example provides a composition for detecting occult blood in excretion, wherein the preparing raw materials were the same as in Example 1 except that cumene hydroperoxide was not added and the resulting absence was made up by hydrogen peroxide.

The preparation method referred to Example 1.

### EXAMPLE 11

This example provides a composition for detecting occult blood in excretion, wherein the preparing raw materials were the same as in Example 1 except that hydrogen peroxide was replaced with an equivalent amount of sodium percarbonate.

The preparation method referred to Example 1.

### EXAMPLE 12

This example provides a composition for detecting occult blood in excretion, wherein the preparing raw materials were the same as in Example 1 except that cumene hydroperoxide was replaced with an equivalent amount of tert-butyl peroxy-2-ethylhexanoate.

The preparation method referred to Example 1.

### EXAMPLE 13

This example provides a composition for detecting occult blood in excretion, wherein the preparing raw materials were the same as in Example 1 except that ethanol was not comprised and the resulting absence was made up by acetone.

The preparation method referred to Example 1.

### EXAMPLE 14

This example provides a composition for detecting occult blood in excretion, wherein the preparing raw materials were the same as in Example 1 except that acetone was not comprised and the resulting absence was made up by ethanol.

The preparation method referred to Example 1.

### EXAMPLE 15

This example provides a composition for detecting occult blood in excretion, wherein the preparing raw materials were the same as in Example 1 except that ethanol was not comprised and the resulting absence was made up by toluene.

The preparation method referred to Example 1.

### Effect Test

The test papers were wetted with the compositions for detecting occult blood in excretion provided in Examples 1-15, respectively, and then dried to obtain detection test papers; after that, the samples (fresh blood from a healthy human body diluted 10000 times with water) were tested, and the color change time and the color retention duration were recorded. The results are as follows:

| Group | Color change time (s) | Color retention duration (day) |
|---|---|---|
| Example 1 | 3 | 30 |
| Example 2 | 3 | 21 |
| Example 3 | 20 | 7 |
| Example 4 | 355 | 3 |
| Example 5 | 595 | 1 |
| Example 6 | 5 | 20 |
| Example 7 | 225 | 3 |
| Example 8 | 335 | 2 |
| Example 9 | 60 | 30 |
| Example 10 | 3 | 0.1 |
| Example 11 | 15 | 1.5 |
| Example 12 | 15 | 1.5 |
| Example 13 | 7 | 22 |
| Example 14 | 12 | 18 |
| Example 15 | 38 | 12 |

It can be found from the above data that the products provided in the present application have the advantages of short color change time and long color retention duration, and can be used for rapid testing of the samples; by comparing Examples 1-5, it can be found that in the present application, by controlling the usage amount of each raw material, the effect of the product can be effectively improved; by comparing Examples 1 and 6-8, it can be found that in the present application, by selecting the specific combinations of benzidine, the color change time can be effectively shortened and the color retention time can be effectively prolonged; by comparing Examples 1 and 9-12, it can be found that in the present application, by selecting the specific peroxide and combinations thereof, the color change time can be effectively shortened and the color retention time can be effectively prolonged; by comparing Examples 1 and 13-15, it can be found that in the present application, by selecting the specific combinations of organic solvents, the color change time can be effectively shortened and the color retention time can be effectively prolonged.

Subsequently, the above test papers were used to test the samples to be tested with different hemoglobin concentrations, and the detection limits of the test papers were analyzed. The results are as follows:

| Group | Detection limit |
|---|---|
| Example 1 | 1:1000000 |
| Example 2 | 1:100000 |
| Example 3 | 1:100000 |
| Example 4 | 1:10000 |
| Example 5 | 1:10000 |
| Example 6 | 1:100000 |
| Example 7 | 1:10000 |
| Example 8 | 1:10000 |
| Example 9 | 1:100000 |
| Example 10 | 1:100000 |
| Example 11 | 1:100000 |
| Example 12 | 1:100000 |
| Example 13 | 1:100000 |
| Example 14 | 1:100000 |
| Example 15 | 1:10000 |

It can be found from the above data that the compositions provided in the present application have high sensitivity, and can effectively detect hemoglobin at a very low content; by comparing Examples 1-5, it can be found that in the present application, by controlling the usage amount of each raw material, the effect of the product can be effectively improved; by comparing Examples 1 and 6-8, it can be found that in the present application, by selecting the specific combinations of benzidine, the detection limit can be effectively reduced and the detection sensitivity can be effectively improved; by comparing Examples 1 and 9-12, it can be found that in the present application, by selecting the specific peroxide and combinations thereof, the detection limit can be effectively reduced and the detection sensitivity can be effectively improved; by comparing Examples 1 and 13-15, it can be found that in the present application, by selecting the specific combinations of organic solvents, the detection limit can be effectively reduced and the detection sensitivity can be effectively improved.

The applicant declares that the composition for detecting occult blood in excretion, the preparation method therefor, and the application thereof in the present application are illustrated by the above examples in the present application, but the present application is not limited to the above examples, that is, the present application does not necessarily rely on the above examples to be implemented. Those skilled in the art should understand that any improvement of the present application, the equivalent substitution of raw materials, the addition of auxiliary ingredients, and the selection of specific methods of the products in the present application shall fall within the protection scope and disclosure scope of the present application.

The optional embodiments of the present application are described in detail above. However, the present application is not limited to the specific details in the above embodiments. A variety of simple variations of the technical solutions in the present application may be carried out within the technical concept of the present application, and these simple variations all fall within the protection scope of the present application.

In addition, it should be noted that without contradiction, the various specific technical features described in the above specific embodiments can be combined in any suitable way, and in order to avoid unnecessary repetition, the various possible combinations are not described in the present application.

1. A composition for detecting occult blood in excretion, comprising:
20-80 parts by weight of an organic solvent,
0.01-50 parts by weight of a thickener,
0.01-2 parts by weight of benzidine,
0.01-5 parts by weight of peroxide,
0.001-0.5 parts by weight of EDTA-2Na, and
10-80 parts by weight of water.

2. The composition according to claim 1, wherein the composition comprises 40-60 parts by weight of the organic solvent, 10-40 parts by weight of the thickener, 0.5-1.5 parts by weight of the benzidine, 1-4 parts by weight of the peroxide, 0.1-0.3 parts by weight of the EDTA-2Na, and 30-60 parts by weight of the water;
the organic solvent comprises any one or a combination of at least two of ethanol, benzene, toluene, pentane, hexane, methanol, ethyl ether, ethyl acetate, acetone, or carbon tetrachloride.

3. The composition according to claim 1, wherein the organic solvent comprises a combination of ethanol and acetone.

4. The composition according to claim 1, wherein the thickener comprises any one or a combination of at least two of xanthan gum, guar gum, pectin, sodium polyacrylate, hydroxypropyl methyl cellulose, hydroxymethyl cellulose sodium, or hydroxymethyl cellulose.

5. The composition according to claim 1, wherein the benzidine comprises any one or a combination of at least two of 4,4′-diaminobiphenyl, tetramethyl benzidine, or 3,3′-dihydroxybenzidine.

6. The composition according to claim 1, wherein the benzidine comprises a combination of 3,3′-dihydroxybenzidine and tetramethyl benzidine.

7. The composition according to claim 1, wherein the peroxide comprises inorganic peroxide and/or organic peroxide.

8. The composition according to claim 7, wherein the peroxide comprises a combination of inorganic peroxide and organic peroxide.

9. The composition according to claim 7, wherein the inorganic peroxide comprises any one or a combination of at least two of hydrogen peroxide, sodium percarbonate, potassium peroxide, calcium peroxide, or barium peroxide.

10. The composition according to claim 7, wherein the inorganic peroxide comprises hydrogen peroxide.

11. The composition according to claim 7, wherein the organic peroxide comprises any one or a combination of at least two of cumene hydroperoxide, diisopropylbenzene hydroperoxide, tert-amyl peroxy-2-ethylhexanoate, or tert-butyl peroxy-2-ethylhexanoate.

12. The composition according to claim 7, wherein the organic peroxide comprises cumene hydroperoxide.

13. The composition according to claim 1, wherein the composition further comprises 0.01-5 parts of a pH-adjusting agent by weight.

14. A preparation method for the composition for detecting occult blood in excretion according to claim 1, which comprises the following steps:
(1) mixing benzidine, peroxide, and an organic solvent to obtain an organic phase;
(2) mixing a thickener, EDTA-2Na, and water to obtain an aqueous phase; and
(3) mixing the aqueous phase with the organic phase to obtain the composition for detecting occult blood in excretion;
steps (1) and (2) are in no particular order.

15. The preparation method according to claim 14, wherein in step (2), mixing the thickener, EDTA-2Na, and water to obtain the aqueous phase further comprises mixing with a pH-adjusting agent;
optionally, in step (3), mixing the aqueous phase with the organic phase further comprises mixing with starch.

16. (canceled)

17. (canceled)

18. The composition according to claim 13, wherein the pH-adjusting agent comprises any one of citric acid, acetic acid, sodium hydroxide, or hydrochloric acid.

19. The composition according to claim 1, wherein the composition further comprises 1-80 parts of starch by weight.

20. The composition according to claim 19, wherein the starch comprises any one or a combination of at least two of corn starch, pea starch, mung bean starch, gelatinized starch, hydroxymethyl starch, crosslinked starch, maltodextrin, oxidized starch, esterified starch, or etherified starch.

21. A method for preparing an occult blood detection device, comprising using the composition for detecting occult blood in excretion according to claim 1.

22. The method for preparing an occult blood detection device according to claim 21, wherein the occult blood detection device comprises any one of a test paper, a test plate, a reagent kit, or a test tube.
